(19) United States
(12) Patent Application Publication
(10) Pub. No.: US 2018/0271914 A1
(43) Pub. Date: Sep. 27, 2018

(54) Methods for Reducing the Extent of Light-induced Tissue Inflammation and Injury

(71) Applicant: Noveome Biotherapeutics, Inc., Pittsburgh, PA (US)

(72) Inventors: David L. Steed, Pittsburgh, PA (US); Kenneth J. Mandell, Arlington, MA (US)

(73) Assignee: Noveome Biotherapeutics, Inc., Pittsburgh, PA (US)

(21) Appl. No.: 15/464,523

(22) Filed: Mar. 21, 2017

(51) Int. Cl. A61K 35/50; A61K 38/19

(52) U.S. Cl. CPC A61K 35/50; A61K 38/19

(57) ABSTRACT

The invention is directed to methods for reducing the extent of light-induced tissue injury. The method comprises applying a therapeutically effective amount of Amnion-derived Cellular Cytokine Solution (ACCS) to the tissue prior to or as soon as possible following exposure to the light. In one particular example, the ACCS is applied within 90 minutes following exposure to the light.

### FIELD OF THE INVENTION

The field of the invention is directed to methods for reducing the extent of light-induced tissue injury resulting from exposure to a light. The method comprises applying a therapeutically effective amount of Amnion-derived Cellular Cytokine Solution (ACCS) to the tissue prior to or as soon as possible following exposure to the light. In one particular embodiment, the ACCS is applied within 90 minutes following exposure to the light.

### BACKGROUND OF THE INVENTION

Light-induced tissue injury can occur when a tissue is exposed to light. Whether tissue damage occurs will be related to a combination of the intensity of the light, its wavelength or frequency, and the length of time the tissue is exposed to the light. Many light-induced tissue injuries are accidental injuries, for example, a welder not wearing proper eye protection while welding. Others are non-accidental and occur, for example, as a result of surgical procedures where light sources are used therapeutically, for example, laser treatment. Other instances occur as a result of unprotected exposures to the sun (i.e., sunburn) or solar eclipses or exposure to the sun on highly reflective snow fields that can lead to direct corneal epithelial injury.

Ultraviolet (UV) light is the most common cause of light-induced injury. Fortunately, the ozone in the atmosphere filters most of the harmful UV wavelengths shorter than 290 nm and natural UV sources, such as the sun, rarely cause injury after short exposures. With respect to UV light exposure to the eyes, the cornea absorbs most of these UV wavelengths. However, it is important to note that UV light damage to the corneal epithelium is cumulative, similar to the effects seen with dermal epithelium due to repeated sunburn.

Artificial sources of UV light also cause eye injuries. As mentioned above, injury from a welder's arc, which is commonly called flash burn, welder's flash, or arc eye, in an example. Other sources of UV light-induced injury include tanning beds, carbon arcs, photographic flood lamps, lightning, electric sparks, and halogen desk lamps.

Prolonged exposure to UV light can lead to chronic solar toxicity, which is associated with several ocular surface disorders (e.g., pinguecula, pterygium, climatic droplet keratopathy, squamous metaplasia, carcinoma). The only ocular cancer associated with UV light exposure is epidermoid carcinoma of the bulbar conjunctiva, which occurs with increased frequency in the tropics and subtropics and has been experimentally replicated in animal models using UV light. Rarely, retinal absorption of visible to near-infrared (400-1400 nm) radiation from welding arcs can lead to permanent, sight-threatening injury.

Prolonged exposure to UV light can also lead to skin cancer. As the ozone layer has been reduced due to greenhouse gases, the incidence of skin cancer has increased as less UV light is being absorbed by the ozone layer.

Another type of light-induced tissue injury occurs when a tissue is exposed to a laser. Laser therapies are very common (see, for example, Zelickson, Z., et al., Dermatol Surg 2014; 40:378-382). Examples include laser hair removal, laser skin resurfacing, tattoo removal, and scar removal. One common side effect is a laser burn on the tissue being treated. These burns can be painful, unsightly and can take 2 weeks or more to heal. Treatment will depend upon the severity of the injury. Typical treatment involves washing the area with mild soap to keep it clean, applying topical antibiotic ointment, and avoiding exposing the burned area to the sun to reduce the chance of hyperpigmentation problems. For severe burns, more involved medical intervention may be required.

In addition, accidental laser-induced injuries are also fairly common, particularly to the eye. General safety precautions include awareness of the risks associated with using lasers. Optical experiments should be carried out on an optical table with all laser beams travelling in the horizontal plane only, and all beams should be stopped at the edges of the table. Users should never put their eyes at the level of the horizontal plane where the beams are in case of reflected beams that leave the table. Adequate eye protection should always be required if there is a significant risk for eye injury. High-intensity beams that can cause fire or skin damage (mainly from class 4 and ultraviolet lasers) and that are not frequently modified should be guided through opaque tubes. Alignment of beams and optical components should be performed at a reduced beam power whenever possible. However, in spite of general safety guidelines, accidental injuries do occur.

Current treatments will depend on the severity of the injury. For skin injuries, the therapy generally consists of washing the area to keep it clean, applying a soothing lotion such as aloe, taking over the counter pain relievers, and monitoring for infection. For injuries to the eyes, the treatments range from antibiotic eye drops, short term steroids and pain medication for corneal injuries to having to perform lens replacement surgery for lens injuries. Currently, no definitive therapy exists for light-induced retinal injuries. Doctors often treat with IV steroids, but this is often not effective and, in some instances, may make the injury worse. It would, therefore, be desirable to have a treatment that could reduce or even eliminate the severity and discomfort resulting from a light-induced tissue injury such as skin injuries that occur following light therapies such as laser therapies, surgical procedures that utilize light therapeutically, and accidental light-induced injuries. It would also be desirable to have a prophylactic treatment that could be applied to a tissue prior to exposure to the light to prevent, or at least reduce, the extent of injury to the tissue. It is an object of the instant invention to provide such a treatment option for this unmet medical need.

### BRIEF SUMMARY OF THE INVENTION

The instant invention provides novel cellular factor-containing solution called Amnion-derived Cellular Cytokine Solution (ACCS) (see U.S. Pat. Nos. 8,058,066 and 8,088,732, both of which are incorporated herein by reference), for use in the described methods for the reduction of light-induced tissue inflammation and injury. Such injuries may be accidental injuries, sunlight-induced injuries such as sunburn, or may be due to the performance of a medical procedure or even as a side effect of a medical procedure. In the case of a medical procedure, it is desirable to apply the composition of the invention prior to and after performance of the procedure to achieve maximum benefit. The route of administration will be dependent upon the tissue and area of the body being treated, as well as the type of procedure itself, but includes topical, intranasal, intradermal, subdermal, subcutaneous, subconjunctival, intravitreal, intraocular administration or any other route of administration deemed appropriate by the attending physician at the time the medical procedure is being performed.

Accordingly, a first aspect of the invention is method for reducing the extent of light-induced tissue inflammation and injury, the method comprising the step of applying a therapeutically effective amount of ACCS to the tissue as soon as possible following exposure to the light.

A second aspect of the invention is method for reducing the extent of light-induced tissue inflammation and injury resulting from exposure to a light, the method comprising the step of applying a therapeutically effective amount of ACCS to the tissue prior to exposure to the light.

In one embodiment of aspects 1 and 2 of the invention, the ACCS is formulated for topical, intranasal, intradermal, subdermal, subcutaneous, subconjunctival, intravitreal, or intraocular administration.

In another embodiment of aspects 1 and 2 of the invention, the ACCS is applied within 90 minutes of exposure to the light.

In another embodiment of aspects 1 and 2 of the invention, the tissue injury is a burn.

In a specific embodiment of aspects 1 and 2 of the invention, the tissue is selected from the group consisting of skin, mucous membrane, cornea, lens and retina. In very specific embodiments, the skin is selected from the group consisting of scarred skin, tattooed skin, and abnormally pigmented skin. In even more specific embodiments, the abnormally pigmented skin is pigmented birthmarks selected from the group consisting of Nevus of Ota, Mongolian spots, Café-au-lait spots, and Nevi. In still other specific embodiments, the abnormally pigmented skin is melisma, hyperpigmentation or hypopigmentation due to skin damage, and vitiligo.

In other embodiments, the skin has vascular birthmarks, wherein the vascular birthmarks are selected from the group consisting of macular stains, hemangiomas, and port wine stains; the skin has psoriatic lesions; or the skin is undergoing exfoliation for hair removal or skin resurfacing.

In a particular embodiment of aspect 1 and 2 of the invention, the light is selected from the group consisting of a laser and a UV light. In specific embodiments, the laser is selected from the group consisting of gas lasers, chemical lasers, dye lasers, metal vapor lasers, solid-state lasers, semiconductor lasers, and UV lasers and the UV light is selected from the group consisting of the sun, a welder's arc, tanning beds, sun lamps, carbon arcs, photographic flood lamps, lightning, electric sparks, and halogen desk lamps.

In a particular embodiment of aspects 1 and 2 of the invention, the ACCS is applied to the tissue before Prostaglandin E₂ (PGE₂) levels increase following exposure to the light.

The above-described aspects and embodiments of the invention are not intended to be limiting, but rather exemplary. Skilled artisans will recognize that additional aspects and embodiments of the invention, though not explicitly or specifically described, are contemplated and encompassed by the teachings and examples set forth in the specification.

### DEFINITIONS

As defined herein “isolated” refers to material removed from its original environment and is thus altered “by the hand of man” from its natural state.

As used herein, the term “protein marker” means any protein molecule characteristic of the plasma membrane of a cell or in some cases of a specific cell type.

As used herein, “enriched” means to selectively concentrate or to increase the amount of one or more materials by elimination of the unwanted materials or selection and separation of desirable materials from a mixture (i.e., separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “extraembryonic cytokine secreting cells” or “ECS cells” means a population of cells derived from the extraembryonic tissue which have the characteristics of secreting a unique combination of physiologically relevant cytokines in a physiologically relevant temporal manner into the extracellular space or into surrounding culture media and which have not been cultured in the presence of any non-human animal-derived products, making them and cell products derived from them suitable for human clinical use. In a preferred embodiment, the ECS cells secrete the cytokines VEGF, Angiogenin, PDGF and the MMP inhibitors TIMP-1 and/or TIMP-2. The physiological range of the cytokine or cytokines in the unique combination is as follows: ˜5-16 ng/mL for VEGF, ˜3.5-4.5 ng/mL for Angiogenin, ˜100-165 μg/mL for PDGF, ˜0.68 μg/mL for TIMP-1 and ˜1.04 μg/mL for TIMP-2.

As used herein, the term “amnion-derived multipotent progenitor cell” or “AMP cell” means a specific population of ECS cells that are epithelial cells derived from the amnion. In addition to the characteristics described above for ECS cells, AMP cells have the following characteristics. They have not been cultured in the presence of any non-human animal-derived products, making them and cell products derived from them suitable for human clinical use. They grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion epithelial cells, from which AMP cells are selected, will have no reaction with an antibody to the stem/progenitor cell marker c-kit (CD117), and minimal to no reaction with an antibody to the stem/progenitor cell marker Thy-1 (CD90).

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the cell, composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such cells, compositions and/or processes.

By the term “serum-free” when referring to certain compositions, growth conditions, culture media, etc., described herein, is meant that no animal-derived serum (i.e., no non-human) is used in the preparation, growth, culturing, expansion, storage or formulation of the cells, composition or process.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium.

As used herein, the term “amnion-derived cellular cytokine solution” or “ACCS” means conditioned medium that has been derived from AMP cells.

As used herein, the term “suspension” means a liquid containing dispersed components, i.e., cytokines. The dispersed components may be fully solubilized, partially solubilized, suspended or otherwise dispersed in the liquid. Suitable liquids include, but are not limited to, water, osmotic solutions such as salt and/or sugar solutions, cell culture media, and other aqueous or non-aqueous solutions.

The term “lysate” as used herein refers to the composition obtained when cells, for example, AMP cells, are lysed and optionally the cellular debris (e.g., cellular membranes) is removed. This may be achieved by mechanical means, by freezing and thawing, by sonication, by use of detergents, such as EDTA, or by enzymatic digestion using, for example, hyaluronidase, dispase, proteases, and nucleases. In some instances, it may be desirable to retain the cellular debris (e.g., cellular membranes), as well.

The term “physiologic” or “physiological level” as used herein means the level that a substance in a living system is found and that is relevant to the proper functioning of a biochemical and/or biological process.

As used herein, the term “substrate” means a defined coating on a surface that cells attach to, grow on, and/or migrate on. As used herein, the term “matrix” or “scaffold” means a three-dimensional (3D) structure that cells grow within or on or that has molecules absorbed into it or adsorbed onto it, that may or may not be defined in its components. It may be composed of biological components, synthetic components, or a combination of both. Further, it may be naturally constructed by cells (i.e., extracellular matrix) or artificially constructed. In addition, the matrix or scaffold may contain components that have biological activity under appropriate conditions.

The term “cell product” or “cell products” as used herein refers to any and all substances made by and secreted from a cell, including but not limited to, protein factors (i.e., growth factors, differentiation factors, engraftment factors, cytokines, morphogens, proteases (i.e., to promote endogenous cell delamination, protease inhibitors), extracellular matrix components (i.e., fibronectin, etc.), lipids, carbohydrates, nucleic acids, etc.

The term “therapeutically effective amount” means that amount of a therapeutic agent necessary to achieve a desired physiological effect (i.e., reducing the extent of light-induced tissue inflammation and injury resulting from exposure to, for example, a laser).

As used herein, the term “pharmaceutically acceptable” means that the components, in addition to the therapeutic agent, comprising the formulation, are suitable for administration to the patient being treated in accordance with the present invention.

As used herein, the term “therapeutic component” means a component of the composition which exerts a therapeutic benefit when the composition is administered to a subject.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

As used herein, the term “tissue” refers to an aggregation of similarly specialized cells united in the performance of a particular function.

As used herein, the terms “a” or “an” means one or more; at least one.

As used herein, the term “adjunctive” means jointly, together with, in addition to, in conjunction with, and the like.

As used herein, the term “co-administer” can include simultaneous or sequential administration of two or more agents.

As used herein, the term “agent” means an active agent or an inactive agent. By the term “active agent” is meant an agent that is capable of having a physiological effect when administered to a subject. Non-limiting examples of active agents include growth factors, cytokines, antibiotics, cells, conditioned media from cells, cells, etc. By the term “inactive agent” is meant an agent that does not have a physiological effect when administered. Such agents may alternatively be called “pharmaceutically acceptable excipients”. Non-limiting examples include time release capsules and the like.

The terms “parenteral administration” and “administered parenterally” are art-recognized and refer to modes of administration other than intranasal, enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, epidural, intracerebral, intraocular, subdural and intrasternal injection or infusion.

As used herein, the term “enteral” administration means any route of drug administration that involves absorption of the drug through the gastrointestinal tract. Enteral administration may be divided into three different categories: oral, gastric, and rectal.

As used herein, the term “topical” administration means a medication that is applied to body surfaces such as the skin, mucous membranes or the ocular surface to treat ailments via a large range of applications including, but not limited to, liquids, sprays, creams, foams, gels, lotions, drops, salves and ointments.

The term “intranasal” or “intranasal delivery” or “intranasal administration” as used herein means delivery within or administered by way of the nasal structures. Such delivery may be targeted delivery to a specific region or regions within the nasal structures.

As used herein, the term “aerosol” means a cloud of solid or liquid particles in a gas.

The terms “particles”, “aerosolized particles”, and “aerosolized particles of formulation” are used interchangeably herein and shall mean particles of formulation comprised of any pharmaceutically active ingredient, preferably in combination with a carrier, (e.g., a pharmaceutically active drug and carrier). As used herein, the term “nebulizer” means a device used to reduce a liquid medication to extremely fine cloudlike particles (i.e., an aerosol). Nebulizers may also be referred to as atomizers and vaporizers.

The terms “sustained-release”, “extended-release”, “time-release”, “controlled-release”, or “continuous-release” as used herein means an agent, typically a therapeutic agent or drug, that is formulated to dissolve slowly and be released over time.

As used herein, the term “inflammation” is defined by redness, swelling, pain and warmth exhibited by a body tissue usually as a result of some insult to the tissue.

As used herein, the term “tissue injury” means the tissue is experiencing or has experienced cell death, loss of tissue function, fibrosis, oncogenesis, DNA damage, and the like.

As used herein, a “laser” refers the type of light source, for example, a collimated and monochromatic light source.

As used herein, the term UV light refers to the wavelengths in the range of 10 nm to 400 nm.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

As used herein, a “wound” is any disruption, from whatever cause, of normal anatomy (internal and/or external anatomy) including but not limited to traumatic injuries such as mechanical (i.e., contusion, penetrating), thermal, chemical, electrical, radiation, light, concussive and incisional injuries; elective injuries such as operative surgery and resultant incisional hernias, fistulas, etc.; acute wounds, chronic wounds, infected wounds, and sterile wounds, as well as wounds associated with disease states (i.e., ulcers caused by diabetic neuropathy or ulcers of the gastrointestinal or genitourinary tract). A wound is dynamic and the process of healing is a continuum requiring a series of integrated and interrelated cellular processes that begin at the time of wounding and proceed beyond initial wound closure through arrival at a stable scar. These cellular processes are mediated or modulated by humoral substances including but not limited to cytokines, lymphokines, growth factors, and hormones. In accordance with the subject invention, “wound healing” refers to improving, by some form of intervention, the natural cellular processes and humoral substances of tissue repair such that healing is faster, and/or the resulting healed area has less scaring and/or the wounded area possesses tissue strength that is closer to that of uninjured tissue and/or the wounded tissue attains some degree of functional recovery.

As used herein the term “standard animal model” refers to any art-accepted animal model in which the compositions of the invention exhibit efficacy.

### DETAILED DESCRIPTION

In accordance with the present invention there may be employed conventional molecular biology, microbiology, and recombinant DNA techniques within the skill of the art. Such techniques are explained fully in the literature. See, e.g., Green et al, 2012, “Molecular Cloning: A laboratory Manual”, Ausubel ed., 2016, “Current protocols in Molecular Biology”, Surzycki et al, 2000, “Basic Techniques in Molecular Biology” Park et al, 2011, “PCR Protocols”, Grandi et al, 2006, “In Vitro Transcription and Translation Protocols”, Anderson ed., 1999, “Nucleic Acid Hybridization”, Alberts et al, 2014, “Molecular Biology of the Cell”, Krebs et al, 2014, “Lewin's Genes XI”, Watson et al, 2014, “Molecular Biology of the Gene”, Nelson et al, 2013, “Lehninger Principles of Biochemistry”, Bonifacino ed., 2016, “Current Protocols in Cell Biology”, Mitry et al, 2012, “Human Cell Culture Protocols”, Helgason et al, 2011, “Basic Cell Culture Protocols”, Guisan et al, 2006, “Immobilization of Enzymes and Cells”, Owen et al, 2012, “Kuby Immunology”, Abbas et al, 2014, “Cellular and Molecular Immunology” Coligan ed., 2016, “Current Protocols in Immunology”.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

### Exemplary Therapeutic Applications

### Injuries to the Eyes

Injuries to the eye can occur from many light sources such as the high intensity light from welding equipment, tanning booths or sunlamps, sunlight, and lasers. For accidental injuries to the ocular surface of the eye, treatment to the affected area should commence as soon as possible following the injury. Treatment may include multiple applications of the composition of the invention, ACCS. Depending on the severity of the injury, treatment may also include multiple applications over several days to achieve the desired therapeutic effect, which is to reduce inflammation and facilitate healing of the injured tissue. Treatment may also include co-administering one or more active agents, for example, an antibiotic, with the composition of the invention. Treatment for light-induced injuries to the lens may ultimately require lens replacement surgery. Treatments for light-induced injuries to the retina are very limited, generally involving IV steroids for severe injuries. The compositions of the invention may be suitable for treating light-induced injuries to the retina via delivery of ACCS to the eyeball itself. This can be achieved by direct injection into the vitreous or, less invasively, through targeted intranasal administration of the ACCS. If it is known in advance that an individual may be at risk of suffering light-induced damage to the eye, for example, during a surgical procedure utilizing light as a therapy, and where protective equipment such as goggles is not feasible, it is desirable to apply the ACCS to the tissue that will be affected by the light prior to exposure. Such prophylactic use will have the effect of minimizing the extent of inflammation and injury and, potentially, preventing inflammation and injury.

### Injuries to the Skin

Injuries to the skin can also occur from many light sources such as tanning booths, sunlamps, and sunlight (each of which can cause sunburn), and lasers. Like injuries to the eye, treatment to the affected skin area should commence as soon as possible following the injury; treatment may include multiple applications of ACCS and, depending on the severity of the injury, treatment may include multiple applications over several days to achieve the desired therapeutic effect. Treatment may also include co-administering one or more active agents, for example, an antibiotic, with the ACCS.

Prophylactic treatment of laser-induced skin injuries is particularly well suited for treatment using the methods of the invention as numerous medical procedures utilize lasers. In addition, applying the ACCS both before and after the procedure could maximize the therapeutic benefit. The following is a non-exhaustive list of medical procedures that utilize lasers and which could benefit from the methods of the invention to reduce inflammation and facilitate healing of light-induced injuries: laser exfoliation for hair removal or skin resurfacing; laser scar removal; laser tattoo removal; laser correction of abnormally pigmented skin including Nevus of Ota, Mongolian spots, Café-au-lait spots, and Nevi, melisma, hyperpigmentation or hypopigmentation due to skin damage, and vitiligo; laser removal of vascular birthmarks including macular stains, hemangiomas, and port wine stains; and laser treatment of psoriatic lesions.

### Injuries to Other Tissues

Also contemplated by the methods of the invention are light-induced injuries to other types of tissue suffering such injuries. Examples include but are not limited to injuries to tissues adjacent to areas inside the body which are being treated during a surgical procedures utilizing laser therapy. Once again, the application of the ACCS could precede the procedure, follow the procedure or be applied both before and after the procedure.

### Compositions and Methods of Making Compositions

Detailed information and methods on the preparation of AMP cell compositions, generation of ACCS, generation of pooled ACCS, detection of cytokines in non-pooled and pooled ACCS using ELISA, and generation of sustained-release compositions can be found in U.S. Pat. Nos. 8,058,066, 8,088,732, 8,278,095 all of which are incorporated herein by reference.

The invention provides for an article of manufacture comprising packaging material and a pharmaceutical composition of the invention contained within the packaging material, wherein the pharmaceutical composition is ACCS. The packaging material comprises a label or package insert which indicates that the ACCS contained therein can be used for therapeutic applications such as, for example, reducing the extent of light-induced tissue inflammation and injury.

### Formulation, Dosage and Administration ACCS

ACCS may be administered to a subject to provide various cellular or tissue functions, for example, reducing the extent of light-induced tissue injury. As used herein “subject” may mean either a human or non-human animal.

The ACCS may be formulated in any conventional manner using one or more physiologically acceptable carriers optionally comprising excipients and auxiliaries. Proper formulation is dependent upon the route of administration chosen. For topical administration, the ACCS compositions may be formulated as a spray, liquid, cream, foam, gel, lotion, salve, drop and ointment, etc. For example, the ACCS may be formulated as a lotion to apply prior to exposure to, for example sunlight, to prevent or minimize sunburn, or as a treatment following sunburn. The compositions may also be administered to the recipient in one or more physiologically acceptable carriers. Carriers for ACCS may include, but are not limited to, solutions of normal saline, phosphate buffered saline (PBS), lactated Ringer's solution containing a mixture of salts in physiologic concentrations, or cell culture medium.

One of skill in the art may readily determine the appropriate dose of the ACCS composition for a particular purpose. An exemplary topical dose is in the range of about 0.1-to-10 milliliters per square centimeter of applied area. One specific exemplary dose is 0.01 milliliters/per square centimeter of applied area. In a particular embodiment, it has been found that a relatively small amount of ACCS is therapeutically useful. One exemplification of such therapeutic utility is the ability for ACCS (including pooled ACCS) to accelerate infected wound healing (for details see U.S. Publication No. 2006/0222634 and U.S. Pat. No. 8,187,881, both of which are incorporated herein by reference). One of skill in the art will also recognize that the number of doses to be administered needs also to be empirically determined based on, for example, severity of injury being treated; patient age, weight, sex, health; other medications and treatments being administered to the patient; and the like. For example, in a specific embodiment, one dose is sufficient to have a therapeutic effect (i.e., reducing the extent of light-induced tissue injury). Other specific embodiments contemplate, 2, 3, 4, or more doses for therapeutic effect. In a specific embodiment, the ACCS is dosed prior to exposure to the light such that it has a prophylactic effect. The frequency of dosing needs to be empirically determined based on similar criteria. In certain embodiments, one dose is administered every day for a given number of days (i.e., once a day for 7 days, etc.). In other embodiments, multiple doses may be administered in one day (every 4 hours, etc.). Multiple doses per day for multiple days are also contemplated by the invention.

In further embodiments of the present invention, at least one additional agent may be combined with the ACCS. Such agents may act synergistically with the compositions of the invention to enhance the therapeutic effect. Such agents include but are not limited to growth factors, cytokines, chemokines, antibodies, inhibitors, antibiotics, immunosuppressive agents, steroids, anti-fungals, anti-virals or cell (i.e., stem cells or stem-like cells, for example, AMP cells). Inactive agents include carriers, diluents, stabilizers, gelling agents, delivery vehicles, ECMs (natural and synthetic), scaffolds, matrices, and the like. When the compositions of the invention are administered conjointly with other pharmaceutically active agents, even less of the compositions may be needed to be therapeutically effective.

The ACCS may also be inserted into a delivery device, e.g., a metered dose device, a syringe, a spray bottle, etc., in different forms. For example, the compositions can be part of a solution contained in such a delivery device. As used herein, the term “solution” includes a pharmaceutically acceptable carrier or diluent. Pharmaceutically acceptable carriers and diluents include saline, aqueous buffer solutions, solvents and/or dispersion media. The use of such carriers and diluents is well known in the art. Preferably, the solution is stable under the conditions of manufacture and storage and may optionally be preserved against the contaminating action of microorganisms such as bacteria and fungi through the use of, for example, parabens, chlorobutanol, phenol, ascorbic acid, thimerosal, and the like. Solutions can be prepared by incorporating the compositions of the invention in a pharmaceutically acceptable carrier or diluent and, as required, other ingredients enumerated above.

The timing of administration of the ACCS will depend upon the type and severity of the light-induced injury. In one embodiment, the ACCS is administered as soon as possible after the injury. In another embodiment, the ACCS is administered more than one time following the injury. In another embodiment, the ACCS is administered prior to the injury (prophylactically) and in still another embodiment, the ACCS is administered both before and after the injury.

Support matrices or scaffolds, including for example membranes and the like, into which the compositions of the invention can be incorporated, absorbed or embedded, or which can have the composition adsorbed onto them, include substances which are recipient-compatible and which degrade into products which are not harmful to the recipient. Detailed information on suitable support matrices, etc. can be found in U.S. Pat. Nos. 8,058,066 and 8,088,732, both of which are incorporated herein by reference. Other suitable matrices and scaffolds are familiar in the art.

A “therapeutically effective amount” of a therapeutic agent within the meaning of the present invention will be determined by a patient's attending physician or veterinarian. Such amounts are readily ascertained by one of ordinary skill in the art and will enable reducing the extent of light-induced tissue injury when administered in accordance with the present invention. Factors which influence what a therapeutically effective amount will be include: the specific activity of the therapeutic agent being used, the extent of the injury, the absence or presence of infection, time elapsed since the injury (whether accidental or as a result of a medical intervention), and the age, physical condition, existence of other disease states, and nutritional status of the patient. Additionally, other medication the patient may be receiving will affect the determination of the therapeutically effective amount of the therapeutic agent to administer.

### Examples

The following examples are put forth so as to provide those of ordinary skill in the art with a complete disclosure and description of how to make and use the compositions and methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Celsius, and pressure is at or near atmospheric.

### Example 1: Ability of ACCS to Reduce the Inflammatory Response Caused by UV Light Exposure

Objective: To determine whether topical application of ACCS significantly reduces the inflammatory response to acute ultraviolet (UV) light delivered on the skin via simulated solar radiation (SSR) in healthy adult volunteers.

Outcome measures: 1) Skin erythema in vivo as quantified by colorimetry; and 2) Levels of thymine dimers (marker of UV light-induced DNA damage) on immunohistochemical staining of skin biopsies.

Protocol: Subjects' buttock skin received 2× the minimum erythema dose on 5 designated 1 inch squares. 1st square or Site 1 did not receive ACCS treatment (control), Sites 2 and 3 were treated immediately with ACCS (immediate treatment), and Sites 4 and 5 were treated with ACCS at a later time point (delayed treatment). Change in erythema compared to non-UV light treated adjacent skin (delta a) was measured at 24, 48 and 72 hours via Minolta Chromometer. Biopsies were done at 24 hours and immunostained for thymine dimer analysis.

Each subject's minimum erythema dose (MED) was calculated according to FDA/Colipa guidelines used for sunscreen testing. This is a quantitative method that utilizes a chromometer that expresses redness as an “a” value. The delta a is the difference between the redness or a value of the UV light-treated spot and an adjacent non-UV light treated normal skin region. Using linear regression analysis, the MED is defined as the dose of UV light that induces a delta of 2.5 units. Once the MED was obtained, each subject was given SSR at 2× their MED on 5 separate areas on the buttocks. These sites were then either 1) untreated, 2) & 3) immediately treated with ACCS, or (4 & 5) treated with ACCS about 6 hours later. ACCS treatment for sites 2-4 continued two times a day for a total of 3 days (72 hours post-SSR). Erythema assessment was performed 24, 48, and 72 hours post-SSR.

Results: There was a statistically significant difference in erythema at 24 (p=0.001), 48 (p=0.003) and 72 (p=0.007) hours between skin sites that were treated immediately with ACCS compared to skin that was not treated after 2× MED of UV light exposure via SSR.

In this particular experiment, delayed treatment with ACCS did not show a significant effect on UV light-induced erythema as there was no statistically significant difference between the delta a of skin that had delayed treatment and skin that had no treatment. Hence, in this experiment, the benefits of ACCS in diminishing inflammation induced by UV light exposure were evident only upon immediate treatment of UV-light treated skin.

ACCS and Cyclopyrimidine dimers (CPDs)—Because UV light-induced erythema has been equated with DNA damage, skin biopsies obtained 24 hours after SSR were immunostained for the presence of cyclopyrimidine dimers, a signature UV light-induced DNA lesion. Biopsies from subjects 6 and 10 were not of good quality which prevented proper staining, so only 8 biopsies were examined. Standard manufacturer-recommended protocols were used for immunostaining and confocal fluorescent microscopy was used for the imaging. The higher the mean fluorescence intensity (A.U.) (y-axis), the greater the levels of CPDs. Quantitation was performed using Metamorph software. The mean fluorescence intensity in untreated skin was 4175 A.U. compared to only 3209 A.U. in ACCS treated skin. This difference is statistically significant (p=0.03).

Conclusions: In this cohort of 10 healthy, light-skinned adults, immediate treatment with ACCS following a dose of UV light equivalent to twice the sunburn threshold, resulted in a decrease in erythema which was visually appreciable and confirmed quantatively via colorimetry. In addition, skin biopsies obtained 24 hours after UV light exposure demonstrated a decrease in cyclopyrimidine dimers in ACCS treated skin compared to untreated skin.

### Example 2: Effect of ACCS in an Animal Model of Laser Exfoliation of Hair

ACCS is tested in an animal model of laser hair removal (see, for example, Zeng, D., et al, Effect of alexandrite laser treatment for hair removal in Tibet mini-pigs, Nan Fang YiKe Da ue Xue Bao 2009 Apr; 29(4):697-700).

Throughout the specification, various publications have been referred to. It is intended that each publication be incorporated by reference in its entirety into this specification

What is claimed is:

1. A method for reducing the extent of light-induced tissue inflammation and injury resulting from exposure to light, the method comprising the step of applying a therapeutically effective amount of Amnion-derived Cellular Cytokine Solution (ACCS) to the tissue following exposure to the light.

2. A method for reducing the extent of light-induced tissue inflammation and injury resulting from exposure to a light, the method comprising the step of applying a therapeutically effective amount of ACCS to the tissue prior to exposure to the light.

3. The method of claim 1 or 2 wherein the ACCS is formulated for topical, intranasal, intradermal, subdermal, subcutaneous, subconjunctival, intravitreal, or intraocular administration.

4. The method of claim 1 or 2 wherein the ACCS is applied within 90 minutes of exposure to the light.

5. The method of claim 1 or 2 wherein the tissue injury is a burn.

6. The method of claim 1 or 2 wherein the tissue is selected from the group consisting of skin, mucous membrane, cornea, lens and retina.

7. The method of claim 6 wherein in the skin is selected from the group consisting of scarred skin, tattooed skin,—abnormally pigmented skin, and skin having psoriatic lesions.

8. The method of claim 6 wherein the skin is undergoing exfoliation or skin resurfacing.

9. The method of claim 1 or 2 wherein the light is selected from the group consisting of a laser and a UV light.

10. The method of claim 9 wherein the laser is selected from the group consisting of gas lasers, chemical lasers, dye lasers, metal vapor lasers, solid-state lasers, semiconductor lasers, and UV lasers.

11. The method of claim 9 wherein the UV light is selected from the group consisting of the sun, a welder's arc, tanning beds, sun lamps, carbon arcs, photographic flood lamps, lightning, electric sparks, and halogen desk lamps.
